(19) United States
(12) Patent Application Publication
(10) Pub. No.: US 2013/0203084 A1
(43) Pub. Date: Aug. 8, 2013

(54) SPECIFIC DETECTION OF HUMAN CHORIONIC GONADOTROPIN BETA SUBUNIT TYPE II PRODUCED BY TROPHOBLASTIC AND NEOPLASTIC CELLS

(71) Applicants: Dominique Bellet, Boulogne-Billancourt (FR); Sophie Richon, L'lsle Adam (FR); Lydia Aldaz-Carroll, Paris (FR); Alain Paul Pecking, Colombes (FR)

(72) Inventors: Dominique Bellet, Boulogne-Billancourt (FR); Sophie Richon, L'lsle Adam (FR); Lydia Aldaz-Carroll, Paris (FR); Alain Paul Pecking, Colombes (FR)

(73) Assignee: Universite Paris Descartes, Paris (FR)

(21) Appl. No.: 13/634,026

(22) PCT Filed: Mar. 11, 2011
(86) PCT No.: PCT/EP11/53676

§ 371 Date: Jan. 18, 2013

Related U.S. Application Data
(60) Provisional application No. 61/282,645, filed on Mar. 11, 2010.

(51) Int. Cl. G01N 33/574

(52) U.S. Cl. CPC G01N 33/57484
USPC 435/7.92; 436/501; 435/7.1; 530/388.24; 530/391.3; 435/451; 435/336; 435/70.21

(57) ABSTRACT

The present invention provides a novel method to distinguish between HCG β type I and type II gene expression using specific antibody. The specific recognition of HCGβ encoded by type II genes and expressed by trophoblastic and neoplastic cells might improve the clinical usefulness of assays aimed at either diagnosing tumors or screening Down's syndrome. The present invention also provides a diagnostic kit for determining the amount of HCGβ type II in a biological sample. The present invention additionally provides process of preparation and screening hybridoma capable of specifically recognizing HCGβ type II and recombinant antibody thereof. Finally, the present invention provides methods for detecting trophoblast or non-trophoblast malignancy in a sample.

The present invention provides a novel method to distinguish between HCGβ type I and type II gene expression using specific antibody. The specific recognition of HCGβ encoded by type II genes and expressed by trophoblastic and neoplastic cells might improve the clinical usefulness of assays aimed at either diagnosing tumors or screening Down's syndrome. The present invention also provides a diagnostic kit for determining the amount of HCGβ type II in a biological sample. The present invention additionally provides process of preparation and screening hybridoma capable of specifically recognizing HCGβ type II and recombinant antibody thereof. Finally, the present invention provides methods for detecting trophoblast or non-trophoblast malignancy in a sample.

Human chorionic gonadotropin (HCG) is a member of the glycoprotein hormone family, which also comprises LH, FSH, and TSH (1). These hormones share a common α-subunit of 92 amino acids that is non-covalently associated with a hormone β-subunit. HCG mediates its action through the LH/HCG receptor, and its major function is to maintain the progesterone production of corpus luteum during early pregnancy. The alpha subunit of HCG lacks HCG activity, but it has been shown to stimulate prolactin production in decidual cells (2) (3). The beta subunit of HCG, which contains 145 amino acids, also lacks HCG activity, but several studies report that it exerts growth-promoting activity (1). In addition to its expression by trophoblastic cells during pregnancy, the HCG β subunit is produced by normal tissues of differing histological origins and is expressed by gonadal and nongonadal neoplasms (4, 5).

The alpha subunit of HCG is encoded by one gene on chromosome 12q21.1-23 (6). The HCGβ subunit however, is encoded by six non-allelic genes (CGB genes). The sequencing of the human genome offers a novel opportunity to check the sequences and organization of these genes clustered on chromosome 19q13.3 and named CGB1 or β1, CGB2 or β2, CGB3 or β3, CGB5 or β5, CGB7 or β7 and CGB8 or β8 (7) (8) (9). Genes β1 and β2 are considered pseudogenes that are not expressed whereas the remaining four genes encode the same protein, with the exception of β7 gene which encodes an alanine at position 117 as opposed to an aspartic acid in the other three genes (8)(10) (11). On the basis of the amino acid residues displayed at position 117, genes encoding the HCGβ subunit were classified as type I genes if they encoded an alanine (β7) or as type II genes if they encoded an aspartic acid (β3, β5, β8). Initially, it was described that normal nontrophoblastic tissues express type I genes whereas, in addition to type I genes, normal trophoblast, malignant trophoblastic and nontrophoblastic tissues of differing histological types express type II genes (5). These differences at the mRNA level open the possibility of a specific distinction at the protein level between the HCGβ subunit expressed by most normal non trophoblastic tissues and the HCGβ subunit produced by normal trophoblast and by malignant cells.

Indeed, immunoassays for HCG and HCG derivatives are important in the diagnosis and monitoring of pregnancies and HCGβ-secreting malignancies, and in testing for Down's syndrome (12, 13). In addition to intact HCG and its free subunits, various molecular forms and fragments of HCG are found in biological fluids (for a review see (1)). Part of the HCG as well as its free β-subunit in urine have intrachain nicks at various positions between amino acids 44 and 48. These forms may also occur in the serum of cancer patients and in patients with trophoblastic disease (14-16). Also, most of the HCG immunoreactivity in urine from pregnant women and cancer patients consist of the beta-core fragment (HCGβcf), which comprises amino acids 6-40 and 55-92 linked by disulphide bridges (for a review see (1)). The development of highly specific and sensitive immunoassays for the detection of free HCGβ has been based on the selection of monoclonal antibodies (mAbs) capable of distinguishing between the different isoforms of HCG. The values obtained from these tests can differ depending on the epitopes and isoforms recognized by the antibodies, for example depending on their sensitivity to the nicked form (17) (18). However, a specific antibody able to distinguish between the free HCGβ subunits translated and transcribed from either type I or type II genes has never been described: fusion experiments using synthetic peptides analogous to the 114-122 region of HCGβ as immunogens and aimed at generating monoclonal antibodies capable of distinguishing an acid aspartic from an alanine at position 117 have been unsuccessful.

Currently there is a need to provide specific methods and reagents to distinguish normal nontrophoblastic tissues expressing type I genes from normal trophoblast, malignant trophoblastic and malignant nontrophoblastic tissues of differing histological types expressing type II genes.

This is the object of the present invention.

Surprisingly, the inventors have demonstrated that mAbs directed to a discontinuous epitope encompassing residues 1 through 7 and 82 through 92 of HCGβ type II, particularly the mAbs named FBT11 and FBT11-II, which are specific to free HCGβ and recognize the nicked form of this subunit are capable of discriminating between HCGβ subunits encoded by type I (HCGβ type I) and type II gene (HCGβ type II).

Thus, in a first aspect, the present invention provides an in vitro method for specifically detecting or quantifying the presence of HCGβ type II subunits in a biological sample from a subject susceptible to contain HCGβ subunits type I and type II, wherein this method implements the use of a monoclonal antibody (mAb) specifically directed to a discontinuous epitope that comprises region 1-7 with a lysine and a proline residue at position 2 and 4 respectively and region 82-92 of HCGβ, preferably said antibody being produced by an hybridoma obtained from a mouse which has been immunized with an antigen comprising at least the fragments 1-7 with a lysine and a proline residue at position 2 and 4 respectively and 82-92 of HCGβ, said hybridoma being selected based on the capability of its secreted mAb to specifically recognize the fragments 1-7 with a lysine and a proline residue at position 2 and 4 of the HCGβ type II.

The term “subject” or “patient” includes mammals, e.g., humans, dogs, cows, horses, kangaroos, pigs, sheep, goats, cats, mice, rabbits, rats, and transgenic non-human animals, human is preferred.

The term “biological samples” includes solid (or biopsy) and body fluid samples. The biological samples used in the method of the present invention may include cells, protein, blood or biological fluids such as bone marrow, ascites fluid or brain fluid (e.g., cerebrospinal fluid). Examples of solid biological samples include samples taken from the placenta rectum, central nervous system, bone, breast tissue, renal tissue, the uterine cervix, the endometrium, the head/neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, and the thymus. Examples of “body fluid samples” include samples taken from the blood, peripheral blood (PB), and peripheral blood stem cell (PBSC), serum, semen, prostate fluid, seminal fluid, urine, saliva, sputum, mucus, bone marrow, lymph, and tears.

Samples for use in the assays of the invention can be obtained by standard methods including venous puncture and surgical biopsy.

In a preferred embodiment, the biological sample used in the methods of the present invention is selected from the group consisting of bone marrow, serum, plasma, blood, lymph, peripheral blood, and peripheral blood stem cell, or cells from the placenta the cancerous or suspected cancerous tissue or solid tumor associated with the presence of abnormal HCGβ of type II or adjacent tissue thereof.

Serum, plasma and whole blood are preferred fluid biological samples and biopsy or tissue sample are preferred for solid samples, particularly tissue samples susceptible of containing tumors or trophoblastic cells, particularly from placenta.

In a preferred embodiment, the present invention provides an in vitro method for specifically detecting or quantifying the presence of HCGβ type II subunits in a biological sample susceptible of containing HCGβ subunits type I and type II, wherein this method implements the use of a mAb selected from the group consisting of:

the mAb FBT-11-II produced by the hybridoma deposited pursuant to and in satisfaction of, the requirements of the Budapest Treaty on the International Recognition of the Deposit of Microorganisms for the Purposes of Patent Procedure with the CNCM (Collection Nationale de Cultures deMicroorganismes, Institut Pasteur, 25 rue du Docteur Roux, F-75724 PARIS Cédex 15) on Mar. 9, 2010 under the number I-4281;

the mAb FBT-11 produced by the hybridoma deposited pursuant to and in satisfaction of, the requirements of the Budapest Treaty on the International Recognition of the Deposit of Microorganisms for the Purposes of Patent Procedure with the CNCM on Oct. 3, 1985 under the number I-489;

a recombinant mAb having a sequence comprising at least the 6 CDRs (Complementary Determining Region) of the mAb FBT-11-II produced by the hybridoma deposited under the number I-4821 or at least the 6 CDRs of the mAb FBT-11 produced by the hybridoma deposited under the number I-489.

“Antibody” includes herein immunoglobulin molecules and the antigen binding fragments of these immunoglobulins.

By antigen-binding fragments it is intended to encompass particularly the fragments Fv, Fab, F(ab′)2, Fab′, scFv, scFv-Fc. These antibody fragments are obtained using conventional techniques well-known to those with skill in the art, and the fragments are screened for utility in the same manner as are intact antibodies.

The hybridoma I-4281 which secretes the FBT-11-II antibody results from successive subcloning cycles of the hybridoma I-489 which secretes the FBT-11 antibody which has been demonstrated by the inventors as being specifically directed to a discontinuous epitope that comprises region 1-7 with a lysine and a proline residue at position 2 and 4 respectively and region 82-92 of HCGβ.

In a preferred embodiment, the present invention provides an in vitro method for specifically detecting or quantifying the presence of HCGβ type II subunits in a biological sample susceptible to contain HCGβ subunits type I and type II, wherein this method comprises the steps of:

a) contacting the biological sample from the subject with an antibody selected from the group consisting of:

a monoclonal antibody (mAb) specifically directed to a discontinuous epitope that comprises region 1-7 with a lysine and a proline residue at position 2 and 4 respectively and region 82-92 of HCGβ,

a mAb specifically directed to a discontinuous epitope that comprises region 1-7 with a lysine and a proline residue at position 2 and 4 respectively and region 82-92 of HCGβ, wherein this antibody is produced by an hybridoma obtained from a mouse which has been prior immunized with an antigen comprising at least the fragments 1-7 with a lysine and a proline residue at position 2 and 4 respectively and 82-92 of HCGβ, said hybridoma being selected based on the capability of its secreted mAb to specifically recognizing the fragments 1-7 with a lysine and a proline residue at position 2 and 4 of the HCGβ type II;

the mAb FBT-11-II produced by the hybridoma I-4281;

the mAb FBT-11 produced by the hybridoma I-489; and

a recombinant mAb having a sequence comprising at least the 6 CDRs of the mAb FBT-11-II produced by the hybridoma deposited under the number I-4281 or at least the 6 CDRs of the mAb FBT-11 produced by the hybridoma deposited under the number I-489,

or a HCGβ type II-binding fragment thereof,
under conditions permitting the binding of said antibody to the HCGβ type II subunits present in said biological sample; and
b) measuring the amount of the complex formed between said antibody bound to the HCGβ type II subunits so as to thereby determine the amount of HCGβ type II in the sample.

In a preferred embodiment, the present invention provides an in vitro method for specifically detecting or quantifying the presence of HCGβ type II subunits in a biological sample susceptible to contain HCGβ subunits type I and type II, wherein this method comprises the steps of:

a) contacting the biological sample from the subject with a capture antibody capable of binding HCGβ type I and type II under conditions permitting the formation of a complex between the antibody and any HCGβ present in the sample;
b) contacting the complex formed with a second antibody (tracer antibody) selected from the group consisting of:

a monoclonal antibody (mAb) specifically directed to a discontinuous epitope that comprises region 1-7 with a lysine and a proline residue at position 2 and 4 respectively and region 82-92 of HCGβ,

a mAb specifically directed to a discontinuous epitope that comprises region 1-7 with a lysine and a proline residue at position 2 and 4 respectively and region 82-92 of HCGβ, wherein this antibody is produced by an hybridoma obtained from a mouse which has been prior immunized with an antigen comprising at least the fragments 1-7 with a lysine and a proline residue at position 2 and 4 respectively and 82-92 of HCGβ, said hybridoma being selected based on the capability of its secreted mAb to specifically recognizing the fragments 1-7 with a lysine and a proline residue at position 2 and 4 of the HCGβ type II;

the mAb FBT-11-II produced by the hybridoma I-4281;

the mAb FBT-11 produced by the hybridoma I-489; and

a recombinant mAb having a sequence comprising at least the 6 CDRs of the mAb FBT-11-II produced by the hybridoma deposited under the number I-4281 or at least the 6 CDRs of the mAb FBT-11 produced by the hybridoma deposited under the number I-489,

or a HCGβ type II-binding fragment thereof,
under conditions permitting the binding of said antibody to the HCGβ type II subunits present in said biological sample; and
c) measuring the amount of the second antibody bound to the complex formed so as to thereby determine the amount of HCGβ type II in the sample.

In a more preferred embodiment, the present invention provides an in vitro method for specifically detecting or quantifying the presence of HCGβ type II subunits in a biological sample susceptible to contain HCGβ subunits type I and type II, wherein:

in step a), the capture antibody is bound to a solid support and the step comprises the removing of any unbound sample from the solid support; and

in step b), the solid support is contacted with the second antibody.

In another preferred embodiment, the present invention provides an in vitro method for specifically detecting or quantifying the presence of HCGβ type II subunits in a biological sample susceptible to contain HCGβ subunits type I and type II, wherein this method comprises the steps of:

a) contacting the biological sample from the subject with a capture antibody capable of binding HCGβ type I and type II under conditions permitting the formation of a complex between the antibody and any HCGβ present in the sample;
b) contacting the complex formed with a second antibody (tracer antibody) selected from the group consisting of:

a monoclonal antibody (mAb) specifically directed to a discontinuous epitope that comprises region 1-7 with a lysine and a proline residue at position 2 and 4 respectively and region 82-92 of HCGβ,

a mAb specifically directed to a discontinuous epitope that comprises region 1-7 with a lysine and a proline residue at position 2 and 4 respectively and region 82-92 of HCGβ, wherein this antibody is produced by an hybridoma obtained from a mouse which has been prior immunized with an antigen comprising at least the fragments 1-7 with a lysine and a proline residue at position 2 and 4 respectively and 82-92 of HCGβ, said hybridoma being selected based on the capability of its secreted mAb to specifically recognizing the fragments 1-7 with a lysine and a proline residue at position 2 and 4 of the HCGβ type II;

the mAb FBT-11-II produced by the hybridoma I-4281;

the mAb FBT-11 produced by the hybridoma I-489; and

a recombinant mAb having a sequence comprising at least the 6 CDRs of the mAb FBT-11-II produced by the hybridoma deposited under the number I-4281 or at least the 6 CDRs of the mAb FBT-11 produced by the hybridoma deposited under the number I-489,

or a HCGβ type II-binding fragment thereof,
under conditions permitting the binding of said antibody to the HCGβ type II subunits present in said biological sample; and
c) measuring the amount of the second antibody bound to the complex formed;
d) measuring in a second portion of the biological sample the amount of the second antibody bound to the complex formed wherein said second antibody used in step d) is an antibody capable of binding to any complex formed between the first antibody and any HCGβ present in the sample; and
e) determining the ratio of HCGβ type II to [HCGβ type I+II (optionally+HGG native if also present)] present in the biological sample from the measurements performed in c) and d).

In a more preferred embodiment, the first antibodies used (capture antibody) are mAbs directed to the carboxyl terminal portion of HCGβ, preferably directed to an epitope comprising at least 6 amino acid residues, preferably at least 10, 12, 15 or 20 residues between the fragment AA118-147 of the HCGβ type I or II, and/or preferably, also directed to an epitope comprising at least 6 amino acid residues, preferably at least 10, 12, or 15 residues between the fragment AA95-116 of the HCGβ type I or II preferably the monoclonal antibodies (mAbs) named FB09 or FB12 which were obtained as previously described (20, 22, 23), or a HCGβ-binding fragment thereof.

MAbs FB09 and FB12, elicited against a synthetic peptide analogous to the COOH 109-145 terminal portion (CTP) of HGGβ, are directed against the 134-139 and 110-116 regions, respectively (23). These mAbs are specific for either HCG or its HCGβ subunit and do not bind to LH or its LHβ subunit.

In a more preferred embodiment, the first antibodies used (capture antibody) for this above method when are a mix of two antibodies (preferably 50% V/V for each type of antibody), one specifically directed against the 134-139 or the 134-140 epitope region (such as FB09 antibody) and the second directed against the 110-116 epitope region (such as FB12 antibody).

The antibody, named FBT10, secreted by the hybridoma deposited under the number I-488 with the CNCM on Oct. 3, 1985 can be also used as capture antibody in the method of the present invention.

In another embodiment, the present invention provides an in vitro method for specifically detecting or quantifying the presence of HCGβ type II subunits in a biological sample susceptible to contain HCGβ subunits type I and type II, wherein this method comprises the steps of:

a) contacting the biological sample from the subject with a capture antibody capable of specifically binding HCGβ type II under conditions permitting the formation of a complex between the antibody and any HCGβ present in the sample, this capture antibody being selected from the group consisting of:

a monoclonal antibody (mAb) specifically directed to a discontinuous epitope that comprises region 1-7 with a lysine and a proline residue at position 2 and 4 respectively and region 82-92 of HCGβ,

a mAb specifically directed to a discontinuous epitope that comprises region 1-7 with a lysine and a proline residue at position 2 and 4 respectively and region 82-92 of HCGβ, wherein this antibody is produced by an hybridoma obtained from a mouse which has been prior immunized with an antigen comprising at least the fragments 1-7 with a lysine and a proline residue at position 2 and 4 respectively and 82-92 of HCGβ, said hybridoma being selected based on the capability of its secreted mAb to specifically recognizing the fragments 1-7 with a lysine and a proline residue at position 2 and 4 of the HCGβ type II;

the mAb FBT-11-II produced by the hybridoma I-4281;

the mAb FBT-11 produced by the hybridoma I-489; and

a recombinant mAb having a sequence comprising at least the 6 CDRs of the mAb FBT-11-II produced by the hybridoma deposited under the number I-4281 or at least the 6 CDRs of the mAb FBT-11 produced by the hybridoma deposited under the number I-489,

or a HCGβ type II-binding fragment thereof;
b) contacting the complex formed with a second antibody (tracer antibody) capable of binding HCGβ type I and type II, and, optionally native HCG, under conditions permitting the formation of a complex between the antibody and any HCGβ present in the sample;
c) measuring the amount of the second antibody bound to the complex formed; and
d) determining the presence of HCGβ type II present in the biological sample from the measurements performed in c).

In a more preferred embodiment, the second antibodies used (tracer antibody) in this above method are mAbs directed to the carboxyl terminal portion of HCGβ, preferably directed to an epitope comprising at least 6 amino acid residues, preferably at least 10, 12, 15 or 20 residues between the fragment AA118-147 of the HCGβ type I or II, and/or preferably, also directed to an epitope comprising at least 6 amino acid residues, preferably at least 10, 12, or 15 residues between the fragment AA95-116 of the HCGβ type I or II preferably the monoclonal antibodies (mAbs) named FB09 or FB12 which were obtained as previously described (20, 22, 23), or a HCGβ-binding fragment thereof.

The antibody, named FBT10, secreted by the hybridoma deposited under the number I-488 with the CNCM on Oct. 3, 1985 can be also used as tracer antibody in the above method of the present invention.

In a more preferred embodiment, the antibodies anti-HCGβ type II. used, particularly as tracer second antibody (or tracer antibody anti-HCGβ type I/II used for determining the ratio HCGβ type II to HCGβ type I and II) can be labelled antibody.

“Labelled antibody” as used herein includes antibodies that are labeled by a detectable means and includes enzymatically, radioactively, fluorescently, chemiluminescently, bioluminescently, biotin or magnetic bead labeled antibodies by any of the many different methods known to those skilled in this art.

One of the ways in which an antibody can be detectably labelled is by linking the same antibody to an enzyme. This enzyme, in turn, when later exposed to its substrate, will react with the substrate in such a manner as to produce a chemical moiety which can be detected, for example, by spectrophotometric, fluorometric or by visual means. Enzymes which can be used to detectably label HCGβ type II-specific antibody (or HCGβ type I/II antibody) include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Detection may be accomplished using any of a variety of immunoassays. For example, by radioactively labelling an antibody, it is possible to detect the antibody through the use of radioimmune assays. A description of a radioimmune assay (RIA) may be found in Laboratory Techniques and Biochemistry in Molecular Biology, by Work T. S. et al., North Holland Publishing Company, NY (1978), with particular reference to the chapter entitled “An Introduction to Radioimmune Assay and Related Techniques” by Chard T. The radioactive isotope can be detected by such means as the use of a gamma counter or a scintillation counter or by audioradiography. Isotopes which are particularly useful for the purpose of the present invention are: ³H, ¹³¹I, ³⁵S, ¹⁴C, and preferably ¹²⁵I.

It is also possible to label an antibody with a fluorescent compound. When the fluorescently labelled antibody is exposed to light of the proper wave length, its presence can then be detected due to fluorescence. Among the most commonly used fluorescent labelling compounds are fluorescein isothiocyanate, rhodamine, phycoerytherin, phycocyanin, allophycocyanin, ophthaldehyde and fluorescamine.

An antibody can also be detectably labelled using fluorescence emitting metals such as ¹⁵²Eu, or others of the lanthanide series. These metals can be attached to the antibody using such metal chelating groups as diethylenetriaminepentaacetic acid (DTPA) or ethylenediaminetetraacetic acid (EDTA).

An antibody can also be detectably labelled by coupling it to a chemiluminescent compound. The presence of the chemiluminescent-tagged antibody is then determined by detecting the presence of luminescence that arises during the course of a chemical reaction. Examples of particularly useful chemiluminescent labelling compounds are luminol, luciferin, isoluminol, theromatic acridinium ester, imidazole, acridinium salt and oxalate ester.

Likewise, a bioluminescent compound may be used to label an antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labelling are luciferin, luciferase and aequorin.

In the detection assays of the invention, the amount of binding of the antibody to the biological sample can be determined by the intensity of the signal emitted by the labelled antibody and/or by the number cells in the biological sample bound to the labelled antibody.

The detection or the level of HCGβ type II in a biological sample may be determined by a radioimmunoassay, an immunoradiometric assay, and/or an enzyme immunoassay.

“Radioimmunoassay” is a technique for detecting and measuring the concentration of an antigen using a labelled (i.e. radioactively labelled) form of the antigen (HCGβ type II). The concentration of HCGβ type II in a biological sample is measured by having the antigen in the sample compete with a labelled (i.e. radioactively) antigen for binding to an antibody to the antigen. To ensure competitive binding between the labelled antigen and the unlabeled antigen, the labelled antigen is present in a sufficient concentration to saturate the binding sites of the antibody. The higher the concentration of antigen in the sample, the lower the concentration of labelled antigen that will bind to the antibody.

In a radioimmunoassay, to determine the concentration of labelled antigen bound to an antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (i.e. covalently) to Sepharose beads, polystyrene wells, polyvinylchloride wells, or microtiter wells. By comparing the concentration of labelled antigen bound to antibody to a standard curve based on samples having a known concentration of antigen, the concentration of antigen in the biological sample can be determined.

An “Immunoradiometric assay” (IRMA) is an immunoassay in which the antibody reagent is radioactively labeled. An IRMA requires the production of a multivalent antigen conjugate by techniques such as conjugation to a protein e.g., rabbit serum albumin (RSA). The multivalent antigen conjugate must have at least 2 epitopes per molecule and these epitopes must be located at a sufficient distance to allow binding by at least two antibodies to the antigen. For example, in an IRMA the multivalent antigen conjugate can be attached to a solid surface such as a plastic sphere.

Unlabelled “sample” antigen and antibody to antigen which is radioactively labelled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

The most common enzyme immunoassay is the “Enzyme-Linked Immunosorbent Assay (ELISA)”. The “Enzyme-Linked Immunosorbent Assay (ELISA)” is a technique for detecting and measuring the concentration of an antigen using a labelled (i.e. enzyme linked) form of the antibody.

In a “sandwich ELISA”, an antibody (i.e. anti-HCGβ) is linked to a solid phase (i.e. a microtiter plate) and exposed to a biological sample containing antigen (i.e. HCGβ type II). The solid phase is then washed to remove unbound antigen. A labelled antibody (i.e. anti-HCGβ type II and enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and 3-galactosidase. The enzyme linked antibody reacts with a substrate to generate a colored reaction product that can be assayed for.

In a “competitive ELISA”, antibody is incubated with a sample containing antigen (i.e. HCGβ type II). The antigen-antibody mixture is then contacted with an antigen-coated solid phase (i.e. a microtiter plate). The more antigen present in the sample, the less free antibody that will be available to bind to the solid phase. A labelled (i.e. enzyme linked) secondary antibody is then added to the solid phase to determine the amount of primary antibody bound to the solid phase.

In an “immunohistochemistry assay” which can be also used in the method of the present invention, a section of tissue (biopsy) is tested for specific proteins by exposing the tissue to antibodies that are specific for the protein that is being assayed. The antibodies are then visualized or quantified by any of a number of methods to determine the presence and amount of the protein present. Examples of methods used to visualize antibodies are, for example, through enzymes linked to the antibodies (e.g., luciferase, alkaline phosphatase, horseradish peroxidase, or P-galactosidase), or chemical methods (e.g., DAB/Substrate chromagen) or gold, fluorescent or labelled antibodies by any of the many different methods known to those skilled in this art.

In a third aspect, the present invention is directed to a kit for specifically detecting or quantifying the presence of HCGβ type II subunits in a biological sample susceptible to contain HCGβ subunits type I and type II, wherein this kit comprises:

a) a monoclonal antibody (mAb) selecting from the group consisting of:

a mAb specifically directed to a discontinuous epitope that comprises region 1-7 with a lysine and a proline residue at position 2 and 4 respectively and region 82-92 of HCGβ;

a mAb specifically directed to a discontinuous epitope that comprises region 1-7 with a lysine and a proline residue at position 2 and 4 respectively and region 82-92 of HCGβ, wherein this antibody is produced by an hybridoma obtained from a mouse which has been prior immunized with an antigen comprising at least the fragments 1-7 with a lysine and a proline residue at position 2 and 4 respectively and 82-92 of HCGβ, said hybridoma being selected based on the capability of its secreted mAb to specifically recognizing the fragments 1-7 with a lysine and a proline residue at position 2 and 4 of the HCGβ type II;

the mAb FBT-11-II produced by the hybridoma deposited pursuant to and in satisfaction of, the requirements of the Budapest Treaty on the International Recognition of the Deposit of Microorganisms for the Purposes of Patent Procedure with the CNCM (Collection Nationale de Cultures de Microorganismes, Institut Pasteur, 25 rue du Docteur Roux, F-75724 PARIS Cédex 15) on Mar. 9, 2010 under the number I-4281;

the mAb FBT-11 produced by the hybridoma deposited pursuant to and in satisfaction of, the requirements of the Budapest Treaty on the International Recognition of the Deposit of Microorganisms for the Purposes of Patent Procedure with the CNCM on Oct. 3, 1985 under the number I-489;

a recombinant mAb having a sequence comprising at least the 6 CDRs (Complementary Determining Region) of the mAb FBT-11-II produced by the hybridoma deposited under the number I-4281 or at least the 6 CDRs of the mAb FBT-11 produced by the hybridoma deposited under the number I-489, optionally, said antibody can be labelled with a dtectable marker,

b) optionally a mAb selected from the group consisting of mAbs directed to the carboxyl terminal portion of HCGβ, preferably directed to an epitope comprising at least 6 amino acid residues, preferably at least 10, 12, 15 or 20 residues between the fragment AA118-147 of the HCGβ type I or II, and, preferably, also directed to an epitope comprising at least 6 amino acid residues, preferably at least 10, 12, or 15 residues between the fragment AA95-116 of the HCGβ type I or II preferably the monoclonal antibodies (mAbs) named FB09 or FB12 which were obtained as previously described (20, 22, 23), optionally labelled with a detectable marker.

In another aspect, the present invention comprises a hybridoma deposited with the CNCM (Collection Nationale de Cultures de Microorganismes, Institut Pasteur, 25 rue du Docteur Roux, F-75724 PARIS Cédex 15) on Mar. 9, 2010 under the number I-4281.

The present invention is also directed to the isolated monoclonal antibody FBT-11-II secreted by the hybridoma deposited with the CNCM (Collection Nationale de Cultures de Microorganismes, Institut Pasteur, 25 rue du Docteur Roux, F-75724 PARIS Cédex 15) on Mar. 9, 2010 under the number I-4281, or HCGβ type II binding fragment thereof.

In another aspect, the present invention concerns a method for the production of a hybridoma cell capable of secreting monoclonal antibodies specifically recognizing the HCGβ type II, wherein said method comprises the step of:

a) immunization of a mammal animal with an immunologically effective amount of an antigen comprising at least the fragments 1-7 with a lysine and a proline residue at position 2 and 4 respectively and 82-92 of HCGβ, optionally with an enhancing carrier preparation;
b) isolating antibodies anti-HCGβ type II producing lymphocytes which do not recognize HCGβ type I from the spleen, lymph nodes or peripheral blood of that mammal animal; and
c) immortalizing these antibodies anti-HCGβ type II producing lymphocytes by fusion of said lymphocytes to cells of same species mammal animal myeloma line.

The present invention also comprises a method for the production of monoclonal antibodies specifically recognizing HCGβ type II, wherein said method comprises the step of:

a) producing a hybridoma cell capable of secreting monoclonal antibodies specifically recognizing HCGβ type II according to the present invention;
b) culturing said hybridoma cell in appropriate culture medium and culture conditions;
c) purifying or isolating from said culture medium the monoclonal antibodies which are secreted.

Hybridoma cells producing a monoclonal antibody of the invention are detected by screening the hybridoma culture supernatants for antibodies that bind HCGβ type II, e.g., using a standard ELISA assay.

A monoclonal antibody can be produced by the following steps. In all procedures, an animal is immunized with an antigen such as a protein (or peptide thereof) as described above for preparation of a polyclonal antibody. The immunization is typically accomplished by administering the immunogen to an immunologically competent mammal in an immunologically effective amount, i.e., an amount sufficient to produce an immune response. Preferably, the mammal is a rodent such as a rabbit, rat or mouse. The mammal is then maintained on a booster schedule for a time period sufficient for the mammal to generate high affinity antibody molecules as described. After a sufficient time to generate high affinity antibodies, the animal (e.g., mouse) is sacrificed and antibody-producing lymphocytes are obtained from one or more of the lymph nodes, spleens and peripheral blood. Spleen cells are preferred, and can be mechanically separated into individual cells in a physiological medium using methods well known to one of skill in the art. The antibody-producing cells are immortalized by fusion to cells of a mouse myeloma line.

Mouse lymphocytes give a high percentage of stable fusions with mouse homologous myelomas; however rat, rabbit and frog somatic cells can also be used. Spleen cells of the desired antibody-producing animals are immortalized by fusing with myeloma cells, generally in the presence of a fusing agent such as polyethylene glycol. Any of a number of myeloma cell lines suitable as a fusion partner can be used with to standard techniques, for example, the P3-NS1/1-Ag4-1, P3-x63-Ag8.653 or Sp2/0-Ag14 myeloma lines, available from the American Type Culture Collection (ATCC), Rockville, Md.

The fusion-product cells, which include the desired hybridomas, are cultured in selective medium such as HAT medium, designed to eliminate unfused parental myeloma or lymphocyte or spleen cells. Hybridoma cells are selected and are grown under limiting dilution conditions to obtain isolated clones. The supernatants of each clonal hybridoma is screened for production of antibody of desired specificity and affinity, e.g., by immunoassay techniques to determine the desired antigen such as that used for immunization. Monoclonal antibody is isolated from cultures of producing cells by conventional methods, such as ammonium sulfate precipitation, ion exchange chromatography, and affinity chromatography (Zola et al., Monoclonal Hybridoma Antibodies: Techniques And Applications, Hurell (ed.), pp. 51-52, CRC Press, 1982).

Hybridomas produced according to these methods can be propagated in culture in vitro or in vivo (in ascites fluid) using techniques well known to those with skill in the art.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies comprising at least the 6 CDRs of the FBT-11 or FBT-11-II can be produced by recombinant DNA techniques known in the art.

In another aspect, the present invention is directed to a method for diagnosis and monitoring of cancers, particularly aggressive cancers by specifically determining or quantifying the presence or absence of HCGβ type II implementing the method of the present invention, the presence or the level of HCGβ type II being correlated with the presence of cancer cells and, optionally to the diagnosis of an aggressive cancer.

The present invention also comprises an in vitro method for determining the subject's response to an anti-cancer therapy, in a subject who is receiving or has received therapy for a state associated with cancer wherein said method comprises the step of the specific detection or of the specifically quantitative measurement of HCGβ type II in a biological sample from the subject by the method according to the present invention.

“Cancer” includes a malignant neoplasm characterized by deregulated or uncontrolled cell growth. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

The term “aggressive” (or “invasive”) as used herein with respect to cancer refers to the proclivity of a tumor for expanding beyond its boundaries into adjacent tissue, or to the characteristic of the tumor with respect to metastasis. Invasive cancer can be contrasted with organ-confined cancer.

Preferably, the present invention is directed to a method for diagnosis and monitoring of trophoblastic or non trophoblastic malignancies including lung, thyroid, prostate, bladder or breast cancers (see 31, 33-36).

In bladder cancers, these genes are predominantly expressed in invasive bladder cancers. In breast cancers, expression of type II genes has prognostic value for relapse-free survival. Moreover, it is well established that the more malignant forms of gestational trophoblastic diseases express excessive amounts of HCGβ (12) and that gonadal tumors might also express HCGβ. Thus, it would be useful to monitor patients with trophoblastic and non trophoblastic tumors for presence of HCGβ present in biological fluids and encoded by type II genes.

Moreover, the expression of type I genes by numerous normal tissues might alter the specific recognition of HCGβ expressed during pregnancy.

Thus, in another aspect, the present invention is directed to a method for the screening of Down's syndrome comprising a step of specifically determining or quantifying the presence or absence of HCGβ type II by the method of the present invention, preferably at first trimester.

The invention is particularly directed to the screening of Down's syndrome by specifically determining or quantifying the presence or absence of HCGβ type II in a sample from in a subject who is either pregnant or suspected of being pregnant, wherein the amount of the early pregnancy associated HCGβ type II specific determination is correlated with the risk of Down's syndrome.

In this aspect, the present invention encompasses a method for the screening of Down's syndrome or for evaluating the risk of Down's syndrome, said method comprising:

a) a step of specifically determining or quantifying the presence or absence of HCGβ type II in a sample from in a subject who is either pregnant or suspected of being pregnant, and
b) a step wherein the determination or the evaluation of the risk Down's syndrome is correlated with the amount of the early pregnancy associated HCGβ type II specific determination is correlated with the risk of Down's syndrome.

The following examples and the figures are given for the purpose of illustrating various embodiments of the invention and are not meant to limit the present invention in any fashion.

### BRIEF DESCRIPTION OF THE FIGURES

FIG. 1. Organization of the CGβ/LHβ gene cluster and amino acid sequences of expressed genes. Only genes CGβ3, CGβ5, CGβ7 and CGβ8 code for the hCGβ subunit. Type I genes code for a mature protein with an arginine residue at amino acid 2, a methionine residue at amino acid 4 and an alanine residue at amino acid 117. Type II genes encode a mature protein with a lysine residue at amino acid 2, a proline residue at amino acid 4 and an aspartic acid at amino acid 117.

FIG. 2. Inhibition of 125I-hCGβ binding to monoclonal antibody FBT-11-II by synthetic peptides corresponding to residues 1 through 7 of type I genes (triangles), type II genes (squares) or LHβ (circles).

FIG. 3. Schematic representation of the oligonucleotide sequence corresponding to residues 2, 4 and 117 of the mature protein of the T24 cell line compared to the JEG-3 cell line (NCBI Reference Sequence: NM_{—}033043.1 (see respectively the SEQ ID NO: 1 and SEQ ID NO: 2 for the Homo sapiens chorionic gonadotropin, beta polypeptide 5 (CGB5) mRNA. and polypeptide sequence). Nucleotide differences are indicated in bold type and the corresponding amino acid residues are indicated below. The numbering (1)-(8) corresponds respectively to the sequences SEQ ID NOs: 10-17.

FIG. 4. Immunocytochemical staining of human choriocarcinoma cell line JEG-3 and human bladder carcinoma cell line T24 with hCGβ specific antibodies FB12 (a), FBT11-II (b) or normal mouse IgG1 control (c). Cells were counterstained with hematoxylin. Photos were taken with an 40× objective.

FIG. 5. Immunocytochemical localization of HCGβ in first, second and third trimester placenta. Formalin-fixed, paraffin-embedded sections of 9-week placenta (top), 17-week (middle) or 39-week (bottom) placenta were stained with HCGβ specific antibodies FB12 (a), FBT11-II (b) or normal mouse IgG1 control (c). Cells were counterstained with hematoxylin. FBT11-II showed staining for HCGβ type II in syncytiotrophoblast (ST), whereas cytotrophoblastic cells (CT) were negative. FB12 showed staining for HCGβ type I+II and HCG in ST and in most CT in early placenta, while mid and late placenta were CT negative. Photos were taken with an 40× objective.

FIGS. 6A-6B. FIG. 6A: Binding of biotinylated FBT11-II to HCGβ immobilized on an ELISA plate by anti-CTP antibodies FB09 and FB12 either alone or in combination. Two different concentrations of HCGβ were used: 9.8 ng/ml (indicated in black) and 47 ng/ml (indicated in white). A representative result is shown. FIG. 6B: Standard curve of the ELISA for HCGβ using mAbs FB09 and FB12 as capture antibodies and biotinylated FBT11-II as indicator. Data are mean results of four independent experiments.

FIG. 7. Localization of FBT11 epitopes on the crystal structure of hCG. Two different views of hCG as a ribbon diagram (Protein Data Bank ID 1HRP) (29) are shown. The alpha subunit is represented in blue and the beta subunit is represented in green. The epitope recognized by mAb FBT11-II spanned residues 1-7 and 82-92 and is indicated by black spheres, residues 2 and 4 are shown in green. As a comparison, type I HCGβ is shown with residues 2 and 4 indicated by red spheres, emphasizing the difference between type I and type II proteins concerning the FBT11-II epitope. Residue 1, while not appearing on the crystal structure, has been added for clarity. This image was obtained using the program Swiss-PdbViewer.

### EXAMPLE 1

### Materials and Methods

### Cell Lines

Human choriocarcinoma cell line JEG-3 was cultured in Eagle's Minimum Essential Medium and human bladder carcinoma cell line T24 was cultured in Dulbecco's MEM (4.5 g/L glucose). All media were supplemented with 10% fetal calf serum and 1× penicillin-streptomycin (Invitrogen, Cergy Pontoise, France).

### Solid-Phase Peptide Synthesis

Synthetic 7-mer peptides corresponding to residues 1 through 7 of the HCGβ subunit were synthesized as previously described (20) by the solid-phase method (21) in an Applied Biosystems Model 430 A peptide synthesizer. The sequences of the peptides were as follows: SKEPLRP (SEQ ID NO: 3) (corresponding to residues 1 through 7 of HCGβ encoded by type II genes β3, β5 and β8); SREMLRP (SEQ ID NO: 4) (corresponding to residues 1 through 7 of HCGβ encoded by type I gene β7); SREPLRP (SEQ ID NO: 5) (corresponding to residues 1 through 7 of the LHβ gene).

### Monoclonal Antibodies

Monoclonal antibody (mAbs) FBT-11-II is an IgG1-Kappa produced by the hybridoma deposited pursuant to and in satisfaction of, the requirements of the Budapest Treaty on the International Recognition of the Deposit of Microorganisms for the Purposes of Patent Procedure with the CNCM (Collection Nationale de Cultures de Microorganismes, Institut Pasteur, 25 rue du Docteur Roux, F-75724 PARIS Cédex 15) on Mar. 9, 2010 under the number I-4281.

The hybridoma I-4281 which secretes the FBT-11-II antibody results from successive subcloning cycles of the hybridoma I-489 also deposited pursuant to and in satisfaction of, the requirements of the Budapest Treaty on the International Recognition of the Deposit of Microorganisms for the Purposes of Patent Procedure with the CNCM on Oct. 3, 1985.

Monoclonal antibodies (mAbs) FB09, FB12 and FBT11-II were obtained as previously described (20, 22, 23). MAbs FB09 and FB12, elicited against a synthetic peptide analogous to the COOH 109-145 terminal portion (CTP) of HGGβ, are directed against the 134-139 and 110-116 regions, respectively (23). These mAbs are specific for either HCG or its HCGβ subunit and do not bind to LH or its LHβ subunit. MAb FBT11 and FBT-11-II secreted elicited against purified HCGβ subunit (CR 129), are directed to a discontinuous epitope that comprises region 1-7 with a lysine and a proline residue at position 2 and 4 respectively and region 82-92 of HCGβ. FBT-11 and FBT-11-II are specific for the HCGβ subunit and do not bind to HCG, LH or its LHβ subunit (20).

Competitive Inhibition Assays with Peptides

Competitive inhibition assays were performed as previously described (20). Briefly, ¹²⁵I labelled HCGβ was employed as the tracer. All experiments were performed in 50 mM phosphate buffer, pH 7.5, containing 154 mM NaCl, 0.02% sodium azide, and 1% bovine serum albumin. First, we determined the dilution of FBT11-II which produced a 50% binding to ¹²⁵I-HCGβ (30,000 cpm) in the absence of peptide. Then, competitive inhibition assays were performed with the defined dilution of antibody. Displacement curves were generated in the presence of increasing concentrations of unlabeled peptides as follows: 100 μl of ¹²⁵I—HCGIβ, 100 μl of monoclonal antibody, and 50 μl of the competitive inhibitor were incubated simultaneously at 4° C. for 18 h. The antigen-antibody complex was then precipitated by adding normal human serum diluted (1:3) in phosphate buffer (100 μl) and 1 ml of 20% polyethylene glycol. After centrifugation, the pellet was counted. Dose-response curves showed a half-maximal inhibitory dose for each molecule tested (ID₅₀).

### Sequencing

RNA obtained from human bladder carcinoma cell line T24 was copied into cDNA with 400 units of SuperScript II RNase H— reverse transcriptase (Life Technologies, California, USA). Two μl of this cDNA were used for 35 cycles of polymerase chain reaction (PCR) with 1.25 units of AmpliTaq Gold from Applied Biosystems (Courtaboeuf, France) with the CG Forward and CG Reverse primers (24) to obtain the CGB insert. The PCR products were purified by electrophoresis on 1% agarose gel using the S.N.A.P. gel purification kit from Invitrogen (Cergy Pontoise, France). Next, the inserts and pcDNA3 plasmid (Invitrogen) were digested with XbaI from New England Biolabs (Frankfurt, Germany) overnight at 37° C. and 19 pmol of plasmid were dephosphorylated using 0.4 units of calf intestinal alkaline phosphatise from Promega (Charbonnières-les-bains, France) following the manufacturer's instructions. After precipitation and ligation of the digested products, sequences were cloned using the TOP10F′ chemically competent E. coli from Invitrogen following the manufacturer's instructions. Fragments were directly sequenced with sequencing primes T7 and Sp6 together with the HCG Forward and HCG Reverse primers using the ABI PRISM Dye Terminator Cycle Sequencing Reaction Kit (PE Biosystems, Courtaboeuf, France) on an ABI PRISM 377 DNA sequencer according to the manufacturer's specifications. Primers were: HCG Forward: 5′-TGTGCTCTAGATCATGACCAAGGATGGAGA TGTTCCAG-3′ (SEQ ID NO: 6); HCG Reverse: 5′-GCACAGTCTAGATTATTGTG GGAGGATCGGG-3′ (SEQ ID NO: 7); T7 (forward): 5′-TAATACGACT CACTATAGGG-3′ (SEQ ID NO: 8); Sp6 (reverse): 5′-GATTTAGGTG ACACTATAG-3′ (SEQ ID NO: 9).

### Immunocytochemical and Immunohistochemical Studies

Indirect immunoperoxydase staining of fixed and permeabilized cells was performed using monoclonal antibodies FB12 and FBT11-II. For immunocytochemical studies on cell lines, cells were grown in a permanox Lab-Tek chamber slide (nunc, Thermo Fisher Scientific, Brebières, France), fixed in 4% PFA in PBS for 20 min at RT and then permeabilized in methanol for 8 min at −20° C. Slides were either then stained immediately or stored at −80° C. For immunohistochemical studies on placentas, placental tissue of first trimester pregnancy was obtained from legal abortion and placental samples from late pregnancy was obtained at term from uncomplicated pregnancy. Use of tissues was approved by the local ethical committee. Tissues obtained were fixed in 4% buffered neutral formalin, dehydrated and embedded in paraffin. Sections, 5 to 6 μm in thickness, were deparaffinised and followed by standard histological techniques. Antibodies were diluted in 1% BSA in PBS and staining was performed using the NovoLink detection system kit (A. Menarini diagnostics, Rungis, France) following the manufacturer's instructions. Between steps, slides were washed twice for 5 min in 50 mM TBS pH 7.6 and once in TBS-0.1% Tween 20. Cells were counterstained with Harris hematoxylin.

### Development of a Two-Site ELISA

Maxisorp nunc plates (Thermo Fisher Scientific, Brebieres, France) were coated with 0.25 μg of monoclonal antibody FB09 and/or 0.25 μg of monoclonal antibody FB12 in 0.1M phosphate buffer pH 7.4, blocked with 1% bovine serum albumin in PBS and incubated with the HCGβ standards (ELSA-FBHCG from CIS Bio International, France; 1 ng CIS=1 mIU 1^{st} IRP WHO 75/551) for 1 h at 37° C. Bound HCGβ was detected with monoclonal antibody FBT11-II coupled with biotin for 1 h at 37° C. (Biotin Labeling Kit —NH₂ from Interchim, Montluçon, France). The plate was then incubated with Immunopure streptavidin Horseradish peroxydase conjugated (Pierce, Thermo Fisher Scientific, Brebieres, France) for 10 min at room temperature (RT). TMB from Pierce was used as the substrate and the absorbance was read at 450 nm. Experiments were done in duplicate. The standard curve was constructed with the HCGβ standards used at increasing concentrations ranging from 0.21-47 ng/ml. Linearity was consistently shown in between run assays. Cell culture supernatants from JEG-3 or T24 cell lines were concentrated 10× using amicon ultra-15 centrifugal filter units (nunc, Thermo Fisher Scientific, Brebieres, France).

### EXAMPLE 2

### Competitive Inhibition Assays with Synthetic Peptides Show FBT11-II Specific Recognition of Type II Genes

Type I and type II genes were described based on the residue difference on position 117.

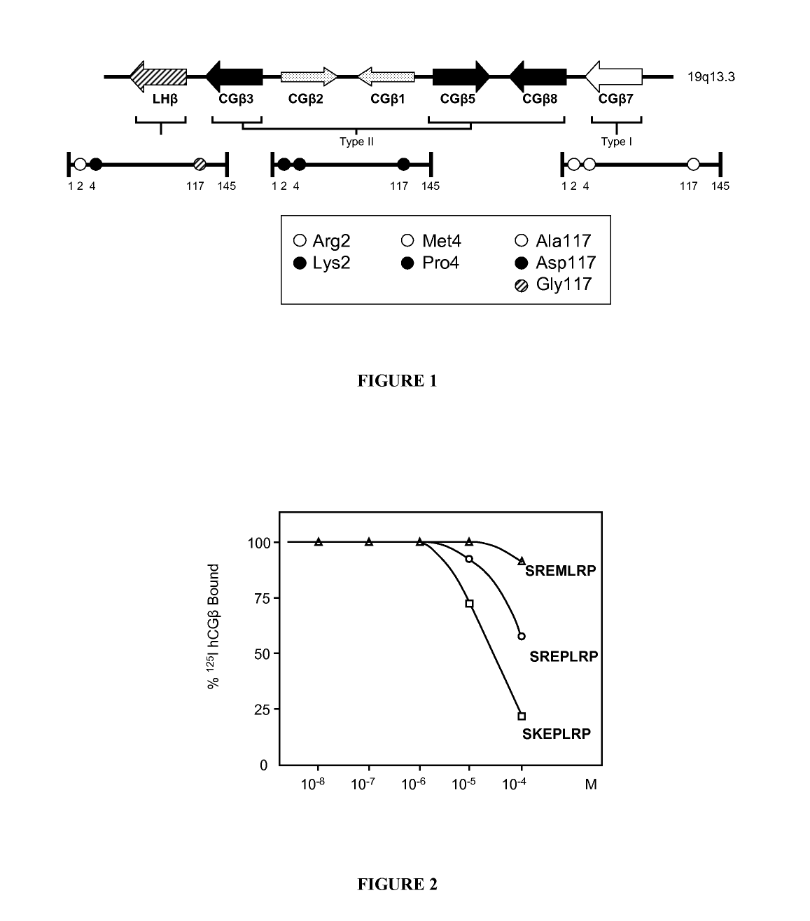

Interestingly, apart from the difference in amino acid 117, two other amino acids differ between type I and type II gene products: Arg2 and Met4 for type I as opposed to Lys2 and Pro4 for type II (11) (9) (FIG. 1). Since it was previously shown that FBT11 recognizes residues 1 through 7 and 82 through 92 of the free HCGβ subunit (20), in this study we examined whether the differences in the N-terminal sequence of HCGβ altered the recognition of FBT11-II. We performed inhibition assays with peptides spanning residues 1 through 7 to determine mAb FBT11-II specificity. We used three peptides analogous to sequences 1-7 of HCGβ encoded by type I gene (SREMLRP, SEQ ID NO: 4), 1-7 of HCGβ encoded by type II genes (SKEPLRP, SEQ ID NO: 3) and 1-7 of LHβ (SREPLRP, SEQ ID NO: 5) (FIG. 2). First, we determined the dilution of FBT11-II which produced a 50% binding to ¹²⁵I-HCGβ in the absence of peptide. Then, competitive inhibition assays were performed with the defined dilution of antibody. Displacement curves were generated in the presence of increasing concentrations of unlabeled peptides.

Synthetic peptide SKEPLRP corresponding to the N-terminal sequence encoded by type II genes exhibits the highest potency in displacing bound ¹²⁵I-HCGβ from antibody FBT11-II. In striking contrast, peptide SREMLRP, differing only in two residues and corresponding to the N-terminal sequence encoded by type I gene, was unable to inhibit the binding of the β-subunit to antibody FBT11-II. Peptide SREPLRP, corresponding to the 1-7 N-terminal sequence of LHβ and displaying only one residue change, was able to inhibit binding of HCGβ to FBT11-II to a lesser degree than SKEPLRP. As FBT11 does not cross react with LHβ (19), these later observations demonstrate that FBT-11 and FBT11-II bind specifically to HCGβ encoded by type II genes.

### EXAMPLE 3

### Sequencing of HCGβ Genes

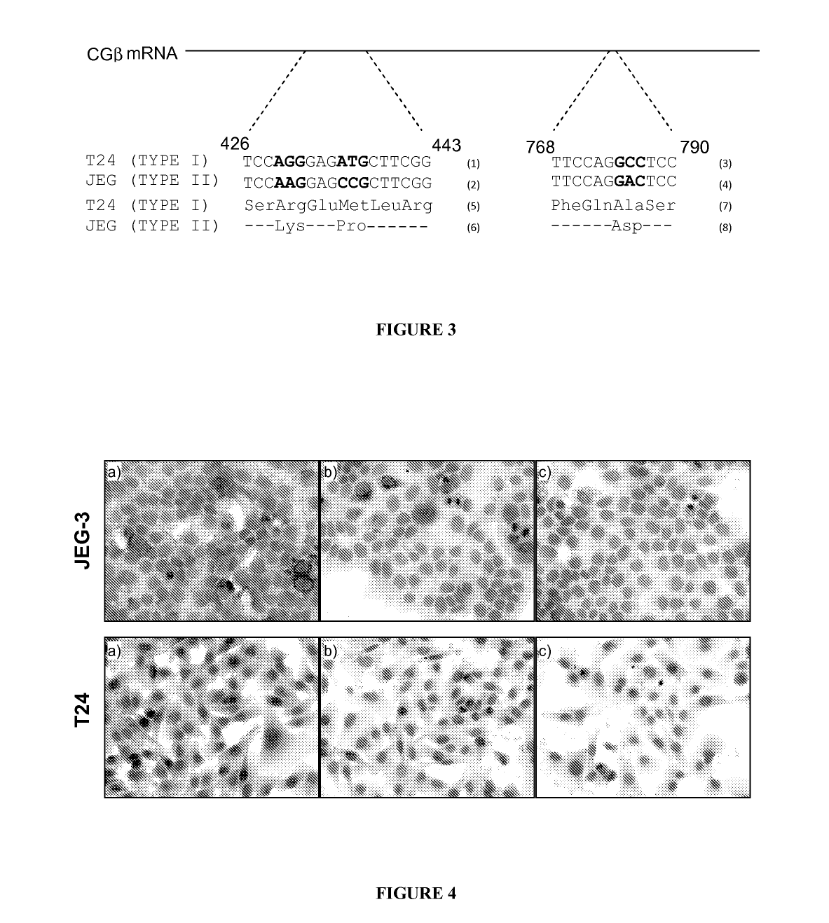

In order to confirm that monoclonal antibody FBT11-II was specific for the HCGβ subunit encoded by type II genes, we selected cell lines which expressed either type I or type II genes as model systems. Human choriocarcinoma cell line JEG-3 expresses preferentially type II genes β5 (25), therefore this cell line was selected as prototypic of cell lines expressing type II genes. It was initially described that the bladder cell line T24 expresses only type I gene with a CG117 index of 0% (5). However, it was later described that this cell line expressed only type II genes (26). In order to clarify this issue, we sequenced the GG beta genes encoded by T24 cell line. The CGB mRNA of human bladder carcinoma cell line T24 was amplified by RT-PCR, cloned and sequenced. We found in three independent experiments that T24 cell line mRNA codes for type I CGB gene β7 (FIG. 3).

### EXAMPLE 4

### FBT11-II Specifically Recognizes HCGβ Encoded by Type II Genes at the Cellular Level

In order to determine whether mAb FBT-11 and FBT11-II specifically recognize HCGβ encoded by type II genes at the cellular level, in situ detection of HCGβ on either human choriocarcinoma cell line JEG-3 or human bladder carcinoma cell line T24 was performed by immunocytochemistry using either mAb FB12 or mAb FBT11-II at 5 μg/ml. Representative results are shown in FIG. 4A. Monoclonal antibody FB12 directed against the 110-116 region is totally specific for HCG and HCGβ, and its binding to HCGβ is unaffected by the presence of an alanine residue instead of an aspartic acid residue at position 117 (23). Thus, FB12 recognizes both type I and type II genes. Monoclonal antibody FBT11 is specific for free HCGβ and had never before been tested for its specific recognition of either type I or type II genes.

As expected, FB12 recognized HCGβ encoded by type II genes expressed by JEG-3 cell line as well as HCGβ encoded by type I gene expressed by T24 cell line (FIG. 4). In contrast, FBT11-II only reacted with HCGβ produced by JEG-3 indicating that these FBT-11 and FBT-11-II antibodies specifically recognize type II genes.

### EXAMPLE 5

### FBT11-II Recognizes HCGβ Encoded by Type II Genes and Produced by Trophoblasts During the Course of Gestation

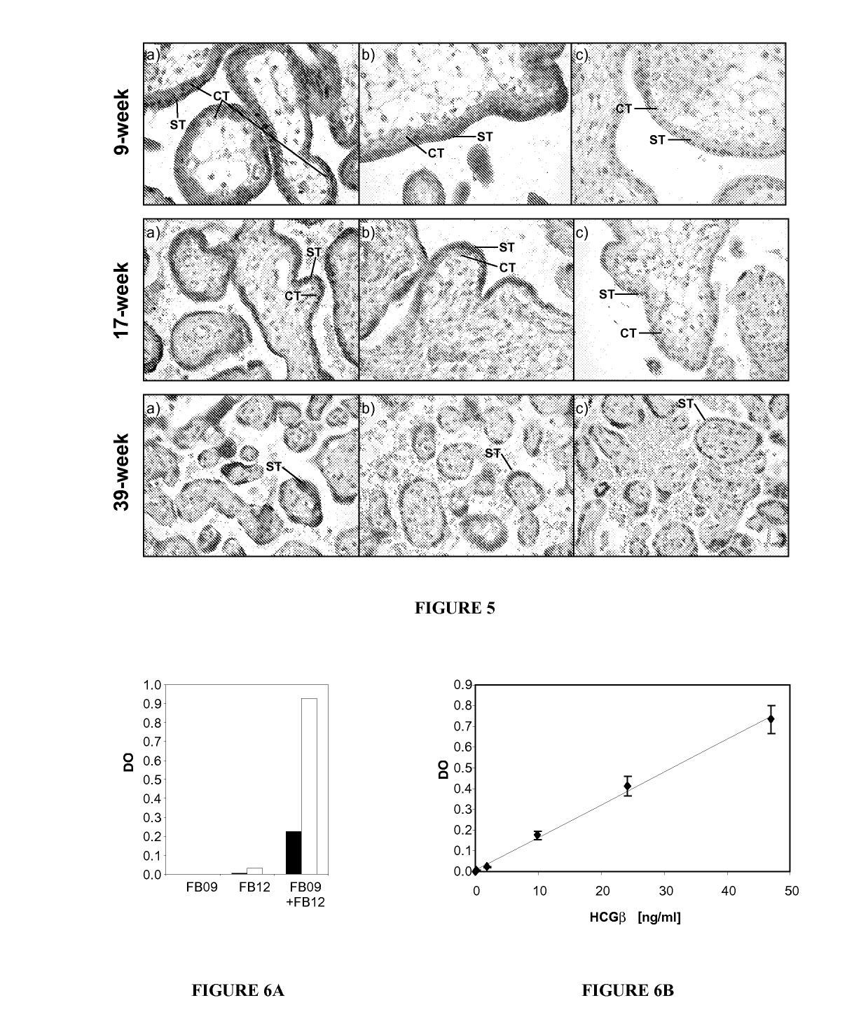

In order to confirm that FBT-11 and FBT11-II are able to recognize in situ HCGβ encoded by type II genes and produced by normal trophoblasts during the course of gestation, we performed immunohistochemical staining on different placenta samples (FIG. 5). In early placenta obtained at 9 weeks, immunostaining with FBT11-II was exclusively localized to syncytiotrophoblast (ST), indicating the presence of HCGβ type II, whereas immunostaining with FB12 was localized to syncytiotrophoblast and to cytotrophoblasts (CT) in most villi, indicating the presence of HCG and/or HCGβ type I and/or type II in these cells. In the case of mid-term placenta (17 weeks) the cytological localization of HCGβ was similar to that in early placenta. In term placenta obtained at 40 weeks of gestation, both immunostaining with FBT11-11 and FB12 were observed in the syncytiotrophoblast.

### EXAMPLE 6

### Development of an ELISA Specific for HCGβ Encoded by Type II Genes

In previous radiolabelled antibody binding experiments based on immunoradiometric assays (IRMAs), it was demonstrated that enhanced binding of indicator antibody to HCG was obtained using anti-CTP antibodies FB09 and FB12 as capture antibodies (23). In order to determine whether this synergistic effect for capturing HCG might also be observed for detecting the HCGβ subunit encoded by type II genes, an enzyme linked immunosorbent assay (ELISA) was designed. This assay is based on mAbs FB09 and FB12, either alone or in combination, to capture HCGβ on a solid phase support and on biotinylated mAb FBT11-II as indicator antibody. The results are shown in FIG. 6A. At both a low dose (9.8 ng/ml) and a high dose of HCGβ (47 ng/ml), no significant binding was found to HCGβ linked to FB09, whereas FB12 slightly bound to HCGβ. By using both FB09 and FB12 (50%, vol/vol), a dramatic synergistic effect was observed. Indeed, a 45 fold increase was observed at 9.8 ng/ml, while a 30 fold increase was detected at 47 ng/ml.

Based on these results, this ELISA was further developed. An HCGβ standard curve is shown in FIG. 6B. The slope of the dose-response curve for HCGβ was 0.99. Intra- and inter-assay standard error of the mean (SEM) were established using a standard HCGβ and were found to be lower than 0.069 for 4 experiments. This assay was used to measure HCGβ in culture supernatants of JEG-3 and T24 cell lines. These cell lines secrete the HCGβ subunit as previously found with an assay based on mAb FBT10. However, mAb FBT10 does not distinguish between HCGβ subunit encoded by type I and type II genes (5). The ELISA based on FBT11-II detected HCGβ present in the supernatant of JEG-3 cell line but did not recognize the free beta subunit secreted by T24 cell line.

The measurement of HCG protein or its variants is important for monitoring pregnancy, for prenatal screening for Down's syndrome and for the diagnosis or follow-up of tumors. Different variants of HCG have been described, including carbohydrate isoforms, nicked variants and truncated versions of the proteins and of individual subunits. Antibodies have been obtained against most of these variants and are used routinely in immunoassays. However, recent reports have demonstrated that because of HCG's heterogeneity, different immunoassays give different results for the same specimens (27). It is important to know the specificity of the method used as the specificity needs to be different for pregnancy and cancer applications. Indeed, during pregnancy, the predominant form of HCG in serum is intact HCG, whereas patients with gestational trophoblastic disease secrete intact HCG, HCGβ and nicked HCG and HCGβ (12). Individuals harbouring nongestational trophoblastic neoplasms, such as germ cell tumors of the testes and ovaries, frequently secrete HCGβ and lesser amounts of HCG, while patients with nontrophoblastic neoplasms secrete only HCGβ.

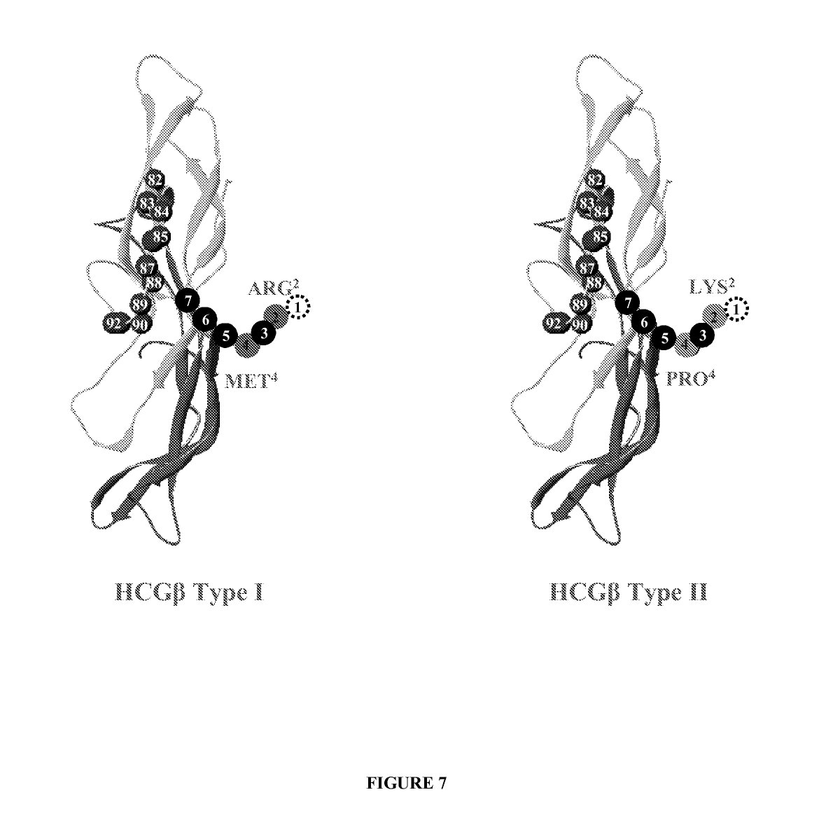

Adding to this complexity, two different HCGβ subunits produced by cells of different origin display several amino acid changes, depending upon the genes expressed by these cells. Type I genes are expressed by cells of nontrophoblastic origin, whereas trophoblastic and malignant nontrophoblastic tissues also express type II genes. HCGβ expressed by type I or II genes differs in three residues located in position 2, 4 and 117 (FIG. 1). It was previously shown that FBT11, an antibody specific against free HCGβ subunit, is able to recognize both nicked and non nicked HCGβ (18) (19). Since FBT11 recognizes a discontinuous epitope comprising residues 1 through 7 and 82 through 92 of HCGβ, this study aimed at determining whether an antibody recognizing this discontinuous epitope of HCGβ (residues 1 through 7 and 82 through 92), such as FBT11 or FBT11-II, could differentiate between type I and type II gene expression. Competitive inhibition assays with peptides show that, in comparison with the peptide corresponding to the type I sequence, only the peptide corresponding to the type II sequence was able to compete for binding of FBT11-II to HCGβ. The peptide corresponding to the 1-7 LHβ amino terminal sequence had a low inhibitory effect on FBT11-II binding to HCGβ, albeit less robust than the type II peptide. As it was demonstrated that FBT11 is totally specific for the free HCGβ subunit and does not bind to free LHβ subunit, the present observations confirm that its specific recognition of HCGβ versus LHβ does not reside within this particular amino terminal region (20). Indeed, FBT11 and FBT11-II recognise a discontinuous region comprising the central 82-92 region of HCGβ in addition to the amino terminal region and it is likely that this central region of HCGβ contributes to the specific recognition of HCGβ over LHβ by FBT11 and FBT11-II. Monoclonal antibodies have been of great use to determine the epitopes on HCGβ (17, 28). In fact, the HCGβ subunit contains at least 13 epitopes, named β1 to β13, of which β1 to β5 are exposed on the HCG heterodimer and β6 and β7 are specific for free HCGβ. The immunodominant structure of this molecule is its core fragment which comprises epitopes β1 to β7 plus four specific HCGβcf determinants whose exact locations have not yet been resolved (β10 to β13). Concerning the precise localization of these epitopes, linear epitopes β8 and β9 have been mapped to residues 109-145, which constitute the C-terminal peptide (CTP) of HCGβ, but all other epitopes are discontinuous. These latter epitopes are located on the cysteine knot (epitope β1) or on the first and third loops protruding from it (epitopes β2 to β6) (17, 28). Each subunit has a cystine-knot motif consisting of two disulfide bonds at its core of extended hairpin loops. A longer loop of double-stranded β-sheets protrudes from one side of the cystine knot, while two shorter hairpin-like loops protrude in the opposite direction. The head-to-tail association of both subunits involves a segment from the 0 subunit that wraps around the α-subunit (29, 30). Considering Berger et al.'s definition of the epitopes on HCGβ (28) it is noteworthy that FBT11 and FBT11-II do not fit into any of these categories. Competitive inhibition experiments performed by RIA (19) have demonstrated that FBT11 recognizes the free HCGβ subunit, but does not recognize total HCG, nor HCGβcf nor LH. Two-site IRMA and two-site immunoenzymmometric assays have shown that FBT11 recognizes the intact and nicked versions of HCGβ (18, 19). We conclude that FBT11 and FBT11-II are directed against a novel and highly specific and discontinuous epitope that comprises region 1-7 with a lysine and a proline residue at position 2 and 4 respectively and region 82-92 of HCGβ (FIG. 7).

To confirm that FBT11 and FBT11-II specifically recognize type II gene expression, T24 and JEG-3 cells were used as prototypic cells expressing HCGβ encoded by either type I or type II genes. First, sequencing of CG genes confirms that the bladder cell line T24 expresses type I gene β7. Indeed, previous reports have shown that tumor progression in bladder tissues is characterized by different patterns of transcription of the CGB genes; type I gene β7 is the only gene transcribed in normal urothelia and Ta tumors whereas, in addition to β7, type II genes were transcribed in T1 to T4 tumors (31). Second, using immunocytochemistry, this paper shows that FBT11 and FBT11-II do not recognize HCGβ encoded by type I gene expressed by T24 cells and only recognizes HCGβ encoded by type II gene expressed by JEG-3 cells. In contrast, antibody FB12 directed against the 110-116 carboxyl terminal portion of HCGβ recognized HCGβ encoded by either type I or type II genes (FIG. 4). Lastly, immunohistochemical staining was carried out on placentas at different times of gestation, showing that FBT11 and FBT11-II consistently stains the syncytiotrophoblast as previously observed (32). As normal trophoblastic cells express type II genes, these results are in line with previous observations.

Studies to differentiate between type I and type II genes have concentrated on elegant techniques using molecular beacons or nested PCR and able to detect a single nucleotide difference, i.e. GCC as opposed to GAC coding respectively for alanine or aspartic acid at position 117 (5, 26). However, depending upon the techniques, different results were observed in tissues and in cell lines as T24. Moreover, these techniques did not exploit the N-terminal differences between type I and type II genes. In order to specifically and easily detect HCGβ encoded by type II genes, a new ELISA was developed to discriminate between type I and type II expression. This ELISA is based on FB09 and FB12 mAbs directed to the carboxyl terminal portion of HCGβ as capture antibodies and on mAb FBT11 and FBT11-II as tracer to bind only to HCGβ encoded by type II genes. To our knowledge, the current study is the first to demonstrate that an assay is specific for HCGβ encoded by type II genes. This assay could be useful to determine the presence or absence of HCGβ encoded by type II genes in biological fluids. While the first assay for HCG was described in 1927, it was stated in 2009 that even though most HCG assays are very reliable there is still a need for better methods for diagnosis and monitoring of cancers (27). Methods that detect HCGβ encoded by type II genes might respond to this need. Indeed, it was shown that type II genes are expressed in many non trophoblastic malignancies including lung, thyroid, prostate, bladder or breast cancers (31, 33-36). In bladder cancers, these genes are predominantly expressed in invasive bladder cancers. In breast cancers, expression of type II genes has prognostic value for relapse-free survival. Moreover, it is well established that the more malignant forms of gestational trophoblastic diseases express excessive amounts of HCGβ (12) and that gonadal tumors might also express HCGβ. Thus, it would be useful to monitor patients with trophoblastic and non trophoblastic tumors for presence of HCGβ present in biological fluids and encoded by type II genes. Moreover, the expression of type I genes by numerous normal tissues might alter the specific recognition of HCGβ expressed during pregnancy. As the measurement of HCGβ is useful for the screening of Down's syndrome at first trimester, it would be preferable to screen for this chromosomal abnormality by using an assay specific for HCG encoded by type II genes. Finally, 83 years after the first description of an HCG assay by Ascheim and Zondek (37), the specific recognition of HCGβ encoded by type II genes might continue to improve the clinical usefulness of such assays.

### REFERENCES

1. Stenman U H, Tiitinen A, Alfthan H, Valmu L, 2006. The classification, functions and clinical use of different isoforms of HCG. Hum Reprod Update 12:769-784.
2. Moy E, Kimzey L, Nelson L, Blithe D, 1996. Glycoprotein hormone alpha-subunit functions synergistically with progesterone to stimulate differentiation of cultured human endometrial stromal cells to decidualized cells: a novel role for free alpha-subunit in reproduction. Endocrinology 137:1332-1339.
3. Blithe D L, Richards R G, Skarulis M C, 1991. Free alpha molecules from pregnancy stimulate secretion of prolactin from human decidual cells: a novel function for free alpha in pregnancy. Endocrinology 129:2257-2259.
4. Marcillac I, Troalen F, Bidart J M, Ghillani P, Ribrag V, Escudier B, Malassagne B, Droz J P, Lhomme C, Rougier P, et al., 1992. Free human chorionic gonadotropin beta subunit in gonadal and nongonadal neoplasms. Cancer Res 52:3901-3907.
5. Bellet D, Lazar V, Bieche I, Paradis V, Giovangrandi Y, Paterlini P, Lidereau R, Bedossa P, Bidart J M, Vidaud M, 1997. Malignant transformation of nontrophoblastic cells is associated with the expression of chorionic gonadotropin beta genes normally transcribed in trophoblastic cells. Cancer Res 57:516-523.
6. Fiddes J C, Goodman H M, 1981. The gene encoding the common alpha subunit of the four human glycoprotein hormones. J Mol Appl Genet 1:3-18.
7. Boorstein W R, Vamvakopoulos N C, Fiddes J C, 1982. Human chorionic gonadotropin beta-subunit is encoded by at least eight genes arranged in tandem and inverted pairs. Nature 300:419-422.
8. Fiddes J C, Goodman H M, 1980. The cDNA for the beta-subunit of human chorionic gonadotropin suggests evolution of a gene by readthrough into the 3′-untranslated region. Nature 286:684-687.
9. Venter J C et al., 2001. The sequence of the human genome. Science 291:1304-1351.
10. Talmadge K, Vamvakopoulos N C, Fiddes J C, 1984 Evolution of the genes for the beta subunits of human chorionic gonadotropin and luteinizing hormone. Nature 307:37-40.
11. Policastro P, Ovitt C E, Hoshina M, Fukuoka H, Boothby M R, Boime I, 1983. The beta subunit of human chorionic gonadotropin is encoded by multiple genes. Journal of Biological Chemistry 258:11492-11499.
12. Stenman U H, Alfthan H, Hotakainen K, 2004. Human chorionic gonadotropin in cancer. Clin Biochem 37:549-561.
13. Wapner R, Thom E, Simpson J L, Pergament E, Silver R, Filkins K, Platt L, Mahoney M, Johnson A, Hogge W A, Wilson R D, Mohide P, Hershey D, Krantz D, Zachary J, Snijders R, Greene N, Sabbagha R, MacGregor S, Hill L, Gagnon A, Hallahan T, Jackson L, 2003. First-trimester screening for trisomies 21 and 18. N Engl J Med 349:1405-1413.
14. Cole L A, Kardana A, Ying F C, Birken S, 1991. The biological and clinical significance of nicks in human chorionic gonadotropin and its free beta-subunit. Yale J Biol Med 64:627-637.
15. Jacoby E S, Kicman A T, Laidler P, Iles R K, 2000. Determination of the Glycoforms of Human Chorionic Gonadotropin {beta}-Core Fragment by Matrix-assisted Laser Desorption/Ionization Time-of-Flight Mass Spectrometry. Clin Chem 46:1796-1803.
16. Kardana A, Elliott M M, Gawinowicz M A, Birken S, Cole L A, 1991. The Heterogeneity of Human Chorionic Gonadotropin (hCG). I. Characterization of Peptide Heterogeneity in 13 Individual Preparations of hCG. Endocrinology 129:1541-1550.
17. Berger P, Sturgeon C, 2008. Human chorionic gonadotropin isoforms and their epitopes: diagnostic utility in pregnancy and cancer. Expert Opinion on Medical Diagnostics 2:1347-1364.
18. Kardana A, Cole L A, 1992. Polypeptide nicks cause erroneous results in assays of human chorionic gonadotropin free beta-subunit. Clin Chem 38:26-33.
19. Bidart J M, Troalen F, Lazar V, Berger P, Marcillac I, Lhomme C, Droz J P, Bellet D, 1992. Monoclonal antibodies to the free beta-subunit of human chorionic gonadotropin define three distinct antigenic domains and distinguish between intact and nicked molecules. Endocrinology 131:1832-1840.
20. Bidart J M, Troalen F, Salesse R, Bousfield G R, Bohuon C J, Bellet D H, 1987. Topographic antigenic determinants recognized by monoclonal antibodies on human choriogonadotropin beta-subunit. J Biol Chem 262:8551-8556.
21. Merrifield R B, 1963. Solid Phase Peptide Synthesis. I. The Synthesis of a Tetrapeptide. Journal of the American Chemical Society 85:2149-2154.
22. Ozturk M, Bellet D, Manil L, Hennen G, Frydman R, Wands J, 1987. Physiological studies of human chorionic gonadotropin (hCG), alpha hC G, and beta hCG as measured by specific monoclonal immunoradiometric assays. Endocrinology 120:549-558.
23. Bellet D H, Ozturk M, Bidart J M, Bohuon C J, Wands J R, 1986. Sensitive and specific assay for human chorionic gonadotropin (hCG) based on anti-peptide and anti-hCG monoclonal antibodies: construction and clinical implications. J Clin Endocrinol Metab 63:1319-1327.
24. Shih 1M, Torrance C, Sokoll L J, Chan D W, Kinzler K W, Vogelstein B, 2000. Assessing tumors in living animals through measurement of urinary beta-human chorionic gonadotropin. Nat Med 6:711-714.
25. Jameson J L, Lindell C M, Habener J F, 1987. Gonadotropin and thyrotropin alpha- and beta-subunit gene expression in normal and neoplastic tissues characterized using specific messenger ribonucleic acid hybridization probes. J Clin Endocrinol Metab 64:319-327.
26. Span P N, Thomas C M, Heuvel J J, Bosch R R, Schalken J A, vd Locht L, Mensink E J, Sweep C G, 2002. Analysis of expression of chorionic gonadotrophin transcripts in prostate cancer by quantitative Taqman and a modified molecular beacon RT-PCR. J Endocrinol 172:489-495.
27. Gronowski A M, 2009. Clinical assays for human chorionic gonadotropin: what should we measure and how? Clin Chem 55:1900-1904.
28. Berger P, Sturgeon C, Bidart J M, Paus E, Gerth R, Niang M, Bristow A, Birken S, Stenman U H, 2002. The ISOBM TD-7 Workshop on hCG and related molecules. Towards user-oriented standardization of pregnancy and tumor diagnosis: assignment of epitopes to the three-dimensional structure of diagnostically and commercially relevant monoclonal antibodies directed against human chorionic gonadotropin and derivatives. Tumour Biol 23:1-38.
29. Lapthorn A J, Harris D C, Littlejohn A, Lustbader J W, Canfield R E, Machin K J, Morgan F J, Isaacs N R, 1994. Crystal structure of human chorionic gonadotropin. Nature 369:455-461.
30. Wu H, Lustbader J W, Liu Y, Canfield R E, Hendrickson W A, 1994. Structure of human chorionic gonadotropin at 2.6 A resolution from MAD analysis of the selenomethionyl protein. Structure 2:545-558.
31. Lazar V, Diez S G, Laurent A, Giovangrandi Y, Radvanyi F, Chopin D, Bidart J M, Bellet D, Vidaud M, 1995. Expression of human chorionic gonadotropin beta subunit genes in superficial and invasive bladder carcinomas. Cancer Res 55:3735-3738.
32. Maruo T, Ladines-Llave C A, Matsuo H, Manalo A S, Mochizuki M, 1992. A novel change in cytologic localization of human chorionic gonadotropin and human placental lactogen in first-trimester placenta in the course of gestation. Am J Obstet Gynecol 167:217-222.
33. Hotakainen K, Lintula S, Jarvinen R, Paju A, Stenman J, Rintala E, Stenman U H, 2007. Overexpression of human chorionic gonadotropin beta genes 3, 5 and 8 in tumor tissue and urinary cells of bladder cancer patients. Tumour Biol 28:52-56.
34. Yokotani T, Koizumi T, Taniguchi R, Nakagawa T, Isobe T, Yoshimura M, Tsubota N, Hasegawa K, Ohsawa N, Baba S, Yasui H, Nishimura R, 1997. Expression of alpha and beta genes of human chorionic gonadotropin in lung cancer. Int J Cancer 71:539-544.
35. Span P N, Manders P, Heuvel J J, Thomas C M, Bosch R R, Beex L V, Sweep C G, 2003. Molecular beacon reverse transcription-PCR of human chorionic gonadotropin-beta-3, -5, and -8 mRNAs has prognostic value in breast cancer. Clin Chem 49:1074-1080.
36. Bieche I, Lazar V, Nogues C, Poynard T, Giovangrandi Y, Bellet D, Lidereau R, Vidaud M, 1998. Prognostic value of chorionic gonadotropin beta gene transcripts in human breast carcinoma. Clin Cancer Res 4:671-676.
37. Ascheim S, Zondek B, 1927. Das Hormon des Hypophysenvorderlappens: testobjekt zum nachweis des hormons. Klin Wochenschr 6:248-252.

1. An in vitro method for specifically detecting or quantifying the presence of HCGβ type II subunits in a biological sample from a subject susceptible of containing HCGβ subunits type I and type II, wherein this method implements the use of a monoclonal antibody (mAb) specifically directed to a discontinuous epitope that comprises region 1-7 with a lysine and a proline residue at position 2 and 4 respectively and region 82-92 of HCGβ, or a HCGβ type II-binding fragment thereof.

2. The in vitro method according to claim 1, wherein this method implements the use of a mAb selected from the group consisting of:
a mAb FBT 11-II produced by the hybridoma deposited with the CNCM (Collection Nationale de Cultures de Microorganismes, Institut Pasteur) on Mar. 9, 2010 under the number I-4281;
a mAb FBT-11 produced by the hybridoma deposited with the CNCM on Oct. 3, 1985 under the number I-489;
a recombinant mAb having a sequence comprising at least the 6 CDRs (Complementary Determining Region) of the mAb FBT-11-II produced by the hybridoma deposited under the number I-4281 or at least the 6 CDRs of the mAb FBT-11 produced by the hybridoma deposited under the number I-489; and
a HCGβ type II-binding fragment thereof.

3. The in vitro method according to claim 1 or claim 2, wherein this method comprises the steps of:
a) contacting the biological sample from the subject with an antibody selected from the group consisting of:
a monoclonal antibody (mAb) specifically directed to a discontinuous epitope that comprises region 1-7 with a lysine and a proline residue at position 2 and 4 respectively and region 82-92 of HCGβ,
a mAb specifically directed to a discontinuous epitope that comprises region 1-7 with a lysine and a proline residue at position 2 and 4 respectively and region 82-92 of HCGβ, wherein this antibody is produced by an hybridoma obtained from a mouse which has been prior immunized with an antigen comprising at least the fragments 1-7 with a lysine and a proline residue at position 2 and 4 respectively and 82-92 of HCGβ, said hybridoma being selected based on the capability of its secreted mAb to specifically recognizing the fragments 1-7 with a lysine and a proline residue at position 2 and 4 of the HCGβ type II;
a mAb FBT-11-II produced by the hybridoma deposited under the number I-4281;
a mAb FBT-11 produced by the hybridoma deposited under the number I-489; and
a recombinant mAb having a sequence comprising at least the 6 CDRs of the mAb FBT-1′-II produced by the hybridoma deposited under the number I-4281 or at least the 6 CDRs of the mAb FBT-11 produced by the hybridoma deposited under the number I-489,
or a or HCGβ type II-binding fragment thereof, under conditions permitting the binding of said antibody to the HCGβ type II subunits present in said biological sample; and
b) measuring the amount of the complex formed between said antibody bound to the HCGβ type II subunits so as to thereby determine the amount of HCGβ type II in the sample.

4. The in vitro method according to claim 1, wherein this method comprises the steps of:
a) contacting the biological sample from the subject with a capture antibody capable of binding HCGβ type I and type II under conditions permitting the formation of a complex between the antibody and any HCGβ present in the sample;
b) contacting the complex formed with a second antibody (tracer antibody) selected from the group consisting of:
a monoclonal antibody (mAb) specifically directed to a discontinuous epitope that comprises region 1-7 with a lysine and a proline residue at position 2 and 4 respectively and region 82-92 of HCGβ,
a mAb specifically directed to a discontinuous epitope that comprises region 1-7 with a lysine and a proline residue at position 2 and 4 respectively and region 82-92 of HCGβ, wherein this antibody is produced by an hybridoma obtained from a mouse which has been prior immunized with an antigen comprising at least the fragments 1-7 with a lysine and a proline residue at position 2 and 4 respectively and 82-92 of HCGβ, said hybridoma being selected based on the capability of its secreted mAb to specifically recognizing the fragments 1-7 with a lysine and a proline residue at position 2 and 4 of the HCGβ type II;
a mAb FBT-11-II produced by the hybridoma deposited under the number I-4281;
a mAb FBT-11 produced by the hybridoma deposited under the number I-489; and
a recombinant mAb having a sequence comprising at least the 6 CDRs of the mAb FBT-11-II produced by the hybridoma deposited under the number I-4281 or at least the 6 CDRs of the mAb FBT-11 produced by the hybridoma deposited under the number I-489,
or HCGβ type II-binding fragment thereof,
under conditions permitting the binding of said antibody to the HCGβ type II subunits present in said biological sample; and
c) measuring the amount of the second antibody bound to the complex formed so as to thereby determine the amount of HCGβ type II in the sample.

5. The in vitro method according to claim 1 specifically detecting or quantifying the presence of HCGβ type II subunits in a biological sample susceptible to contain HCGβ subunits type I and type II, wherein this method comprises the steps of:
a) contacting the biological sample from the subject with a capture antibody capable of binding HCGβ type I and type II under conditions permitting the formation of a complex between the antibody and any HCGβ present in the sample;
b) contacting the complex formed with a second antibody (tracer antibody) selected from the group consisting of:
a monoclonal antibody (mAb) specifically directed to a discontinuous epitope that comprises region 1-7 with a lysine and a proline residue at position 2 and 4 respectively and region 82-92 of HCGβ,
a mAb specifically directed to a discontinuous epitope that comprises region 1-7 with a lysine and a proline residue at position 2 and 4 respectively and region 82-92 of HCGβ, wherein this antibody is produced by an hybridoma obtained from a mouse which has been prior immunized with an antigen comprising at least the fragments 1-7 with a lysine and a proline residue at position 2 and 4 respectively and 82-92 of HCGβ, said hydridoma being selected based on the capability of its secreted mAb to specifically recognizing the fragments 1-7 with a lysine and a proline residue at position 2 and 4 of the HCGβ type II;
a mAb FBT-11-II produced by the hybridoma deposited under the number I-4281;
a mAb FBT-11 produced by the hybridoma deposited under the number I-489; and
a recombinant mAb having a sequence comprising at least the 6 CDRs of the mAb FBT-11-II produced by the hybridoma deposited under the number I-4281 or at least the 6 CDRs of the mAb FBT-11 produced by the hybridoma deposited under the number I-489,
or HCGβ type II-binding fragment thereof,
under conditions permitting the binding of said antibody to the HCGβ type II subunits present in said biological sample; and
c) measuring the amount of the second antibody bound to the complex formed;
d) measuring in a second portion of the biological sample the amount of the second antibody bound to the complex formed wherein said second antibody used in step d) is an antibody capable of binding to any complex formed between the first antibody and any HCGβ present in the sample; and
e) determining the ratio of HCGβ type II to [HCGβ type I+II (optionally+HGG native if also present)] present in the biological sample from the measurements performed in c) and d).

6. The in vitro method according to claim 4 or claim 5, wherein:
in step a), the capture antibody is bound to a solid support and the step comprises the removing of any unbound sample from the solid support; and—in step b), the solid support is contacted with the second antibody.

7. The in vitro method according to claim 3, wherein the first antibodies used in step a) (capture antibody) are mAbs directed to the carboxyl terminal portion of HCGβ, preferably directed to an epitope comprising at least 6 amino acid residues of the fragment AA 118-147 of the HCGβ type I or II, and directed to an epitope comprising at least 6 amino acid residues AA95-116 of the HCGβ type I or II, or a HCGβ-binding fragment thereof.

8. The in vitro method according to claim 7 the said first antibodies used are the monoclonal antibodies (mAbs) named FB09 or FB12.

9. The in vitro method according to claim 1, wherein the antibodies anti-HCGβ type II used as a tracer second antibody are labelled antibodies.

10. The in vitro method according to claim 1, wherein the method which is implemented to detect or quantify the presence HCGβ type II is an ELISA and/or an immunohistochemistry assay.

11. The in vitro method according to claim 1 wherein the biological sample is a serum/plasma sample or a biopsy from the subject to be tested.

12. A kit for specifically detecting or quantifying the presence of HCGβ type II subunits in a biological sample susceptible to contain HCGβ subunits type I and type II, wherein this kit comprises:
a) a monoclonal antibody (mAb) selecting from the group consisting of:
a mAb specifically directed to a discontinuous epitope that comprises region 1-7 with a lysine and a proline residue at position 2 and 4 respectively and region 82-92 of HCGβ;
a mAb specifically directed to a discontinuous epitope that comprises region 1-7 with a lysine and a proline residue at position 2 and 4 respectively and region 82-92 of HCGβ, wherein this antibody is produced by an hybridoma obtained from a mouse which has been prior immunized with an antigen comprising at least the fragments 1-7 with a lysine and a proline residue at position 2 and 4 respectively and 82-92 of HCGβ, said hybridoma being selected based on the capability of its secreted mAb to specifically recognizing the fragments 1-7 with a lysine and a proline residue at position 2 and 4 of the HCGβ type II;
a mAb FBT-11-II produced by the hybridoma deposited with the CNCM (Collection Nationale de Cultures de Microorganismes, Institut Pasteur) on Mar. 9, 2010 under the number I-4281;
a mAb FBT-11 produced by the hybridoma deposited with the CNCM on Oct. 3, 1985 under the number I-489; and
a recombinant mAb having a sequence comprising at least the 6 CDRs (Complementary Determining Region) of the mAb FBT-11-II produced by the hybridoma deposited under the number I-4281 or at least the 6 CDRs of the mAb FBT-11 produced by the hybridoma deposited under the number I-489,
or a HCGβ type II-binding fragment thereof,
optionally, said antibody can be labelled with a detectable marker, and
b) optionally a mAb selected from the group consisting of:
a mAb directed to the carboxyl terminal portion of HCGβ; a mAb directed to an epitope comprising at least 6 amino acid residues of the fragment AA118-147 of the HCGβ type 1 or II; a mAb directed to an epitope comprising at least 6 amino acid residues AA95-116 of the HCGβ type I or II;
a mAb named FB09; and
a mAb named FB12,
or HCGβ-binding fragment thereof,
optionally labelled with a detectable marker.

13. A method for the production of a hybridoma cell capable of secreting monoclonal antibodies specifically recognizing the HCGβ type II, wherein said method comprises the step of:
a) immunization of a mammal animal with an immunologically effective amount of an antigen comprising at least the fragments 1-7 with a lysine and a proline residue at position 2 and 4 respectively and 82-92 of HCGβ, optionally with an enhancing carrier preparation;
b) isolating antibodies anti-HCGβ type II producing lymphocytes which do not recognize HCGβ type I from the spleen, lymph nodes or peripheral blood of that mammal animal; and
c) immortalizing these antibodies anti-HCGβ type II producing lymphocytes by fusion of said lymphocytes to cells of same species mammal animal myeloma line.

14. The Hybridoma deposited with the CNCM (Collection Nationale de Cultures de Microorganismes, Institut Pasteur) on Mar. 9, 2010 under the number I-4281.

15. A method for the production of monoclonal antibodies specifically recognizing HCGβ type II, wherein said method comprises the step of:
a) producing a hybridoma cell according to the method of claim 13 capable of secreting monoclonal antibodies specifically recognizing HCGβ type II according to the present invention;
b) culturing said hybridoma cell in appropriate culture medium and culture conditions;
c) purifying or isolating from said culture medium the monoclonal antibodies which are secreted.

16. An isolated monoclonal antibody FBT-11-II secreted by the hybridoma deposited with the CNCM (Collection Nationale de Cultures de Microorganismes, Institut Pasteur) on Mar. 9, 2010 under the number I-4281.

17. A method for the diagnosis or the monitoring of cancers from biological sample of a subject wherein the specific presence or absence of HCGβ type II is determined by the method according to claim 1, and wherein the diagnosis or the monitoring of said cancer is correlated with the level of HCGβ type II determined in the biological sample tested.

18. The method according to claim 17, wherein said cancers are trophoblastic or non trophoblastic malignancies, optionally selected from the group of lung, thyroid, prostate, bladder or breast cancers.

19. A method for detecting trophoblast or non-trophoblast malignancy m a biological sample comprising the step of:
a) specifically determining the level of the presence or absence of HCGβ type II in said sample by employing the method of claim 1;
b) the presence of HCGβ type II in said sample being correlating the presence of HCGβ type II in said sample with the presence of a trophoblast or non-trophoblast malignancy.

20. A method for the screening of Down's syndrome or for evaluating the risk of Down's syndrome, said method comprising:
a) specifically determining or quantifying the presence or absence of HCGβ type II in a sample from in a subject who is either pregnant or suspected of being pregnant, and
b) correlating the presence or absence of HCGβ type II in said sample with the risk of Down's syndrome.


SEQUENCE LISTING (17 sequences)

SEQ ID NO: 1 - DNA, 880 bp, homo sapiens
  misc_feature: Chorionic gonadotropin, beta polypeptide 5 (CGB5)
tccagcactttgctcgggtcacggcctcctcctggctcccaggaccccaccataggcagaggcaggccttcctacaccctactccctgtgcctccaggctcgactagtccctagcactcgacgactgagtctctgagatcacttcaccgtggtctccgcctcacccttggcgctggaccagtgagaggagagggctggggcgctccgctgagccactcctgcgcccccctggccttgtctacctcttgccccccgaagggttagtgtcgagctcaccccagcatcctacaacctcctggtggccttgccgcccccacaaccccgaggtataaagccaggtacacgaggcaggggacgcaccaaggatggagatgttccaggggctgctgctgttgctgctgctgagcatgggcgggacatgggcatccaaggagccgcttcggccacggtgccgccccatcaatgccaccctggctgtggagaaggagggctgccccgtgtgcatcaccgtcaacaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggactcctcttcctcaaaggcccctccccccagccttccaagtccatcccgactcccggggccctcggacaccccgatcctcccacaataaaggcttctcaatccgca

SEQ ID NO: 2 - protein, 165 aa, homo sapiens
  misc_feature: Chorionic gonadotropin, beta polypeptide 5 (CGB5)
MEMFQGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 3 - protein, 7 aa, Artificial
  Synthetic 7-mer peptides corresponding to residues 1 through 7 of HCGbeta encoded by type II genes beta3, beta5 and beta8
SKEPLRP

SEQ ID NO: 4 - protein, 7 aa, artificial
  Synthetic 7-mer peptides corresponding to residues 1 through 7 of HCGbeta encoded by type I gene beta7
SREMLRP

SEQ ID NO: 5 - protein, 7 aa, artificial
  Synthetic 7-mer peptides corresponding to residues 1 through 7 of the LHbeta gene
SREPLRP

SEQ ID NO: 6 - DNA, 38 bp, artificial
  Primer HCG Forward
tgtgctctagatcatgaccaaggatggagatgttccag

SEQ ID NO: 7 - DNA, 31 bp, artificial
  Primer HCG Reverse
gcacagtctagattattgtgggaggatcggg

SEQ ID NO: 8 - DNA, 20 bp, artificial
  Primer T7 Forward
taatacgactcactataggg

SEQ ID NO: 9 - DNA, 19 bp, artificial
  Primer Sp6 Reverse
gatttaggtgacactatag

SEQ ID NO: 10 - DNA, 18 bp, homo sapiens
  misc_feature: homo sapiens CG beta type I from T24 cell line (from position 426-443)
tccagggagatgcttcgg

SEQ ID NO: 11 - DNA, 18 bp, homo sapiens
  misc_feature: homo sapiens CG beta type II from JEG cell line (from position 426-443)
tccaaggagccgcttcgg

SEQ ID NO: 12 - DNA, 12 bp, homo sapiens
  misc_feature: homo sapiens CG beta type I from T24 cell line (from position 768-790)
ttccaggcctcc

SEQ ID NO: 13 - DNA, 12 bp, homo sapiens
  misc_feature: homo sapiens CG beta type II from JEG cell line (from position 768-790)
ttccaggactcc

SEQ ID NO: 14 - protein, 6 aa, homo sapiens
  misc_feature: homo sapiens CG beta type I from T24 cell line corresponding to mRNA position 426-443
SREMLR

SEQ ID NO: 15 - protein, 6 aa, homo sapiens
  misc_feature: homo sapiens CG beta type II from JEG cell line corresponding to mRNA position 426-443
SKEPLR

SEQ ID NO: 16 - protein, 4 aa, homo sapiens
  misc_feature: homo sapiens CG beta type I from T24 cell line corresponding to mRNA  position 768-790
FQAS

SEQ ID NO: 17 - protein, 4 aa, homo sapiens
  misc_feature: homo sapiens CG beta type II from JEG cell line corresponding to mRNA position 768-790
FQDS